(19) United States
(12) Patent Application Publication
(10) Pub. No.: US 2007/0207226 A1
(43) Pub. Date: Sep. 6, 2007

(54) Composition for enhancing muscle mass development in animals

(76) Inventors: Daniel B. Mowrey, Springville, UT (US); Nathalie Chevreau, Salt Lake City, UT (US); Louis Rinaldi, Deer Mountain, UT (US)

Correspondence Address: HOLME ROBERTS & OWEN, LLP, 299 SOUTH MAIN, SUITE 1800, SALT LAKE CITY, UT, 84111, US

(21) Appl. No.: 11/368,270

(22) Filed: Mar. 3, 2006

(51) Int. Cl. A61K 36/10; A61K 36/81; A61K 31/4439

(52) U.S. Cl. 424/762; 424/773; 424/777; 514/356; 514/343

(57) ABSTRACT

Compositions containing nicotine and/or nicotinic acetylcholine receptor agonists are provided for administration in therapeutically effective amounts to enhance muscle development in animals, the nicotine and/or nicotinic acetylcholine receptor agonists being particularly derived from natural sources that produce beneficially high amounts nicotine and/or nicotinic acetylcholine receptor agonists which is also well-tolerated and ingestible for the intended purpose.

### BACKGROUND OF THE INVENTION

1. Field of the Invention

This invention relates generally to compositions useful for development of muscle mass in animals, and specifically relates to such compositions, particularly dietary supplements, which contain well-tolerated and ingestible nicotine and/or nicotinic acetylcholine receptor agonists in therapeutically effective amounts to enhance muscle mass development in animals.

2. Description of Related Art

Researchers have shown that administration of nicotine or nicotinic acetylcholine receptor (nAChR) agonists have beneficial effects in producing angiogenesis and vasculogenesis, particularly in wound healing events. Nicotine and nAChR agonists have also been shown to have beneficial effects in neuromuscular development. That is, experimental data has demonstrated that administration of pharmaceutical grade nicotine or nAChR agonists dissolved in water can, for example, stimulate certain growth factors, such as fibroblast growth factor (FGF), which facilitates proliferation of vascular endothelial cells in vitro. Other experimental data has shown that pharmaceutical grade nicotine dissolved in water and administered to pregnant rats has a positive effect on improving muscle strength in the neonatal offspring.

It has further been disclosed in pending U.S. Published Application No. 2004/0167179 A1 that pharmaceutical grade nicotine or nAChR agonists, when dissolved in water and administered in connection with exercise, has an enhancing effect on recruiting and mobilizing endogenous stem cells to a specific muscle mass for development of that muscle area or mass that is subjected to exercise. It is suggested that the beneficial effects of administering pharmaceutical grade nicotine or nAChR agonists in the described manner are relevant to those who, in particular, are desirous of improving muscle mass or body tone as a result of weight lifting or similar exercise.

The pharmaceutical grade nicotine and nAChR agonists that have been used in these past endeavors, while providing the intended effect, have been shown to be intolerable for human and animal consumption because of the inherent unpleasant taste and non-comestibility of the pharmaceutical grade nicotine and nAChR agonists. This is particularly significant since the primary mode of nicotine absorption takes place through the membranes or lining of the mouth. Consequently, the fact that pharmaceutical grade nicotine and nAChR agonists are not well-tolerated or suited for human and animal consumption undermines any beneficial effect that may be derived from administering nicotine and nAChR agonists.

Thus, it would be advantageous in the art to provide compositions containing therapeutically effective amounts of nicotine and nAChR agonists for enhancing the development of muscle mass in both humans and animals which is well tolerated and comestibly suitable for ingestion by humans and animals so that they might derive the beneficial effects thereof.

### BRIEF SUMMARY OF THE INVENTION

In accordance with the present invention, compositions are provided which contain nicotine and/or nicotinic acetylcholine receptor agonists in therapeutically effective amounts for providing enhanced muscle mass development in animals, where the nicotine an/or nicotinic acetylcholine receptor agonists are derived from natural sources that provide an improved tolerance and ingestibility of the nicotine to thereby render the compositions tolerable for ingestion by both humans and animals.

The compositions of the present invention contain nicotine and/or nicotinic acetylcholine receptor (nAChR) agonists which are derived from sources that render the nicotine and nAChR agonists more well-tolerated for consumption, and thereby more ingestible. The nicotine and/or nicotinic acetylcholine receptor (nAChR) agonists are particularly derived from natural plant sources that may be referred to herein as “green source.” Green source nicotine and/or nicotinic acetylcholine receptor (nAChR) agonists render a composition that is more palatable and more tolerated when ingested, and can provide increased concentrations of nicotine than are available through use of pharmaceutical grade nicotine and/or nicotinic acetylcholine receptor (nAChR) agonists, such as has been used in the prior art.

While nicotine and/or nicotinic acetylcholine receptor (nAChR) agonists are found in a number of plant sources, it was discovered by the inventors that not all plant sources provide nicotine and/or nicotinic acetylcholine receptor (nAChR) agonists which are well-tolerated and ingestible or of the highest extracted concentration. It has been found that nicotine and/or nicotinic acetylcholine receptor agonists from certain green sources disclosed herein provide not only a more well-tolerated nicotine or nAChR agonist extract, but provide improved concentrations of those materials.

The compositions of the present invention further comprise additional ingredients that may enhance the ingestibility of the nicotine and/or nicotinic acetylcholine receptor (nAChR) agonists by not only enhancing the tolerability of the nicotine and/or nicotinic acetylcholine receptor (nAChR) agonists, but by increasing the effectiveness of the nicotine and/or nicotinic acetylcholine receptor (nAChR) agonists in enhancing muscle mass development.

### DETAILED DESCRIPTION OF THE INVENTION

The compositions of the present invention comprise a blend of botanical ingredients where the principal active ingredient is nicotine and/or nicotinic acetylcholine receptor (nAChR) agonists that is derived from selected plant sources (i.e., green source) determined to provide nicotine and/or nAChR agonists that are well-tolerated and ingestible. Nicotine and nAChR agonists can be found in a number of plant sources, particularly those plants that are in the Solanaceae family, such as tobacco and tomatoes.

Plants in the genus Nicotiana, which includes tobacco, are those sources that most readily come to mind in terms of producing nicotine. However, it is nicotine and nAChR agonists derived from those sources that have been used in the past to effect angiogenesis, vasculogenesis and neuromuscular development, and such nicotine and nAChR agonists have proven to be poorly tolerated at ingestion. Because of their characteristic of poor tolerance and uningestibility, the use of such nicotine and nAChR agonists from the genus Nicotiana has proven to limit their usefulness in administration for, among other things, enhancing muscle mass development.

Thus, it has been discovered that certain other genera, species and cultivars of the Solanaceae family provide sources for deriving nicotine and nAChR agonists that are more well-tolerated and ingestible, and which, in some cases, provide a higher concentration of extracted nicotine and nAChR agonists than can be derived from the traditionally-used or known plant sources. Another source of well-tolerated nicotine or nAChR agonists is the horsetail plant, which is in the Equistetaceae family.

It has been determined, for example, that nicotine and nAChR agonists derived from the genus Lycopersicon (tomatoes), Solanum (potatoes and eggplant) and Physalis (tomatillo), all in the Solanaceae family, are particularly well-tolerated and ingestible. More particularly, it has been determined that nicotine and nAChR agonists derived from green tomatoes is not only well-tolerated and ingestible, but provides an increased concentration of nicotine and nAChR agonists upon extraction from that plant source.

An exemplar process by which nicotine and nAChR agonists may be derived from any of the identified plant sources is as follows:

Extraction Process

Raw plant material (e.g., green tomato or potato) that has been dried by any conventional process, including air drying or placement in a dehydrator, is processed to a dry flake form. From between 5.0 Kg to 10.0 Kg of dried raw plant material is then weighed and placed in a milling machine for processing to a selected size (e.g., about 0.2 mm to about 1.2 mm, or from about 15 mesh to 35 mesh). The milled product is then hydrated using a solvent (e.g., 50% ethanol-water mixture). The hydrated material is then macerated in a stainless steel macerator for a period of from about one hour to six hours, during which time a solid-liquid extraction occurs providing a mother liquor and extracted liquid. The extracted liquid is then distilled to remove or condense the alcohol content in the liquid, and approximately 40% to 70% of the volume of extracted liquid is retained. The resulting distillate is then mixed with the mother liquor to produce a final liquid extract and the liquid extract is placed in a 20.0 liter capacity container. The liquid extract is placed in a drying chamber and dried for a period sufficient to produce a dried powder having a water content of 5% or less. The resulting powder extract derived from the drying process is then tested to assure that it contains from 4 to 7 μg of nicotine or nAChR agonist, and preferably about 6 μg of nicotine or nAChR agonist.

In compositions of the present invention, the nicotine and nAChR agonists extracts may be blended with other phytonutrients or extracts including antioxidants, such as grape seed extract, green tea polyphenols, beta carotenes, vitamin C ascorbic acid and derivatives thereof, Vitamin A retinols and derivatives thereof, Vitamin E tocopherols and tocotrienols and derivatives thereof, oligomeric procynidins and derivatives thereof, carotenoids (e.g., lyopene, lutein, zeaxathine, etc.) resveratrol, carnosic acids and derivatives thereof, and ellagic acid and derivatives thereof.

The compositions may also include anti-inflammatory agents, such as quercetin, rutin, white willow bark (salicylic acid), essential fatty acids (EFA) such as omega-3 fatty acids from vegetable sources or marine sources, hyssop (Arnica montana), scutellaria baicalensis and acacia catechu, and combinations thereof, alfalfa, ashwaganda (withania somnifera), autumn crocus (colchicum autumnale), barberry (berberis vulgaris), beta-sitosterol, bitter orange (citrus aurantium), bittersweet nightshade (solanum dulcamara), boldo (peumus boldus), buchu (agathosma betulina), cat's claw (uncaria tomentosa), cinnamon, comfrey (symphytum officinale), devil's claw (harpagophytum procumbens), dong quai (angelica sinensis), emblica (emblica officinalis), Emu oil (dromaius novae-hollandiae), fenugreek, frankincense (boswellia serrata), ginger, horse chestnut (aesculus hippocastanum), kalanji (nigella sativa), kalmegh (andrographis paniculata), milk thistle (silybum marianum), ocgacosanol, papaya, propolis (propolis balsam), poria (poria cocos), perilla frutescens, Royal jelly, savory (Satureja hortensis), sour cherry (prunus cerasus), tinospora cordifolia and witch hazel (hamamelis viginiana). The anti-oxidant and anti-inflammatory extracts may be beneficial in ameliorating certain effects of more strenuous exercise on muscles when the compositions of the present invention are administered to humans and animals in the development of muscle mass.

The compositions of the present invention may also include one or more vitamins, including but not limited to retinol, thiamin, riboflavin, niacin, pyridoxine, ascorbic acid and Vitamin D. The compositions may also include one or more minerals, such as calcium, iron, magnesium, selenium and zinc.

In further formulation of the compositions, various ingredients may be included in the compositions to improve the aesthetic qualities of the compositions, such as taste, color and ingestibility. Such ingredients would include, for example, flavorings, colorants, binders, fillers, flow agents and lubricating agents. These agents are well-known in the pharmaceutical and dietary supplement industries for formulating compositions into various forms, including tablets, capsules, powders and the like.

An exemplar formula for the present composition may comprise the following:

| INGREDIENT | Percent by weight |
|---|---|
| Nicotine/nAChR agonist extract | 3-9% |
| (e.g., from green tomato extract | |
| containing from 3-7 μg of nicotine or | |
| NAChR agonist) | |
| Antioxidant(s) | 0.001-0.10% |
| (e.g., grape seed extract 95%) | |
| Anti-inflammatory agent(s) | 0.001-0.10% |
| (e.g., quercetin dihydrate, rutin) | |
| Flavoring agents | 75-92% |
| Vitamins and/or minerals | 1-5% |
| Lubricating agents | 1-3% |
| Binding agents | 0.1-1% |
| Flow agents | 0.1-1% |
| Colorants | 0.1-1% |

The foregoing exemplar formulation is not intended to limit the compositions of the present invention. Other ingredients may be added to, and indeed may be eliminated from, the general formula set forth above and still be within the scope of the invention as set forth in the claims. It is requisite to the invention, however, to provide a therapeutically effective amount of nicotine or nAChR agonists in the formula to provide enhanced development of muscle mass.

One exemplary and suitable composition directed to enhancement of muscle mass in humans comprises a 200 mg tablet formulated as follows:

| | | |
|---|---|---|
| Nicotine/nAChR agonist extract (containing about | 3-9 | mg |
| 6 μg of nicotine/nAChR agonist | | |
| Antioxidants (green tea and grape seed extracts) | 0.001-0.002 | mg |
| Anti-inflammatory agents (quercetin and rutin) | 0.001-0.002 | mg |
| Tomato concentrate from tomato paste (flavoring | 20-27 | mg |
| agent) | | |
| Sucrose (primarily a binding agent and flavoring | 110-130 | mg |
| agent) | | |
| Other flavoring agents (bitter masking flavor, | 2-3 | mg |
| Prosweet flavoring agent) | | |
| Sodium chloride | 4-8 | mg |
| Vegetable flavor FG-3294 | 0.2-1.0 | mg |
| Riboflavin (B2) | 12-22 | mg |
| Stearic acid | 4-8 | mg |
| Magnesium stearate | 0.5-1.5 | mg |
| Silicon dioxide | 0.5-1.5 | mg |
| FD&C red #40 lake | 0.5-1.5 | mg |

Although the foregoing exemplar composition may be formed into a tablet form, capsule forms and powdered forms are also within the scope of the invention and are within the skill in the art to form by known methods. However, by way of example, tablets comprising the ingredients noted above may be formulated by mixing the ingredients, all of which are in a dry powder form, in a ribbon blender until well blended. The resulting mixture is then compressed to a specified weight (e.g., 200 mg), a specified thickness (e.g., 0.135 inches) and specified hardness (e.g., 1118-25 Kp) using a suitable tablet press.

Tablets or capsules of the composition of the present invention may further be formed or formulated for timed- or delayed-release by, for example, providing a suitable outer or intermediary coating on or in the tablet or capsule, in known manner, to provide a selected time release of the composition. Any suitable means known in the art by which tablets and capsules may be formed or formulated for timed- or delayed-release may be used.

As previously noted, the compositions of the present invention are particularly suitable for use in enhancing muscle mass development in humans and animals. The enhancement of muscle mass development is mediated, in particular, by administration of the compositions of the present invention in conjunction with the performance of exercise. Thus, administration of a dosage of the composition prior to the performance of exercise has been shown to be effective for increasing muscle mass development (as described, for example, in U.S. Published Application No. 2004/0167179 A1).

For humans, a daily dosage of the compositions of the present invention containing a therapeutically effective amount of nicotine and/or nAChR agonist extract is from three to nine mg. The daily dosage is preferably administered thirty to sixty minutes before commencement of an exercise session. The daily dosage is taken, preferably, by placing the dosage form (e.g., tablet, capsule, powder) under the tongue or in the side of the mouth, allowing the composition to be absorbed slowly in the mouth. The compositions of the present invention may be particularly beneficial when taken in conjunction with the performance of very rigorous exercise (i.e., at or near the exhaustion of a given muscle mass).

The present compositions may also be administered to animals for the enhancement of muscle mass development. For smaller animals, such as dogs or other similarly sized animals (e.g., weighing from about 30 to about 180 pounds or more), a daily dosage of a therapeutically effective amount of nicotine and/or nAChR agonist extract is from about 20 mg to about 35 mg, or from between about 10 μg and 38 μg of nicotine and/or nAChR agonist. For larger animals, such as horses or similarly sized animals (e.g., weighing greater than about 700 pounds), a daily dosage of a therapeutically effective amount of nicotine and/or nAChR agonist extract is from about 45 mg to about 100 mg, or from between about 22 μg and 112 μg of nicotine and/or nAChR agonist. Such daily dosages may be administered to animals in conjunction with the imposition of an exercise regimen, most especially a rigorous regimen, for the subject animal to improve muscle mass development to overcome injuries or to train the animal for improved performance, such as for racing.

The formulas and constituent ingredients described herein are merely examples of various formulations for the compositions of the present invention and are not intended to limit the scope of the invention. Those of skill in the art will understand that the compositions of the present invention, in addition to providing the requisite therapeutically effective amount of nicotine and/or nAChR agonist extract as described herein, may include other ingredients to facilitate the formulation of the compositions.

1. A composition for enhancing muscle mass development in animals, comprising a therapeutically effective amount of nicotine and/or nicotinic acetylcholine receptor agonist extract, the extract containing from about 3 μg to about 7 μg of nicotine and/or nicotinic acetylcholine receptor agonist, and the extract being derived from a natural plant source that provides well-tolerated and ingestible nicotine and/or nicotinic acetylcholine receptor agonist.

2. The composition of claim 1 wherein said natural plant source that provides well-tolerated and ingestible nicotine and/or nicotinic acetylcholine receptor agonist is a plant of the Solanaceae family.

3. The composition of claim 2 wherein said plant source is green tomato.

4. The composition of claim 2 wherein said plant source is eggplant.

5. The composition of claim 2 wherein said plant source is potato plant.

6. The composition of claim 1 wherein said natural plant source that provides well-tolerated and ingestible nicotine and/or nicotinic acetylcholine receptor agonist is the horsetail plant.

7. The composition of claim 1 wherein said daily dosage of nicotine and/or nicotinic acetylcholine receptor agonist extract is from between about 3 mg and about 9 mg in a composition formulated for administration to humans.

8. The composition of claim 1 wherein said daily dosage of nicotine and/or nicotinic acetylcholine receptor agonist extract is from between about 45 mg and about 100 mg in a composition formulated for administration to large animals.

9. The composition of claim 1 wherein said daily dosage of nicotine and/or nicotinic acetylcholine receptor agonist extract is from between about 20 mg and about 35 mg in a composition formulated for administration to small animals.

10. The composition of claim 1 further comprising at least one ingredient selected from the group comprising vitamins and minerals.

11. The composition of claim 1 further comprising at least one antioxidant.

12. The composition of claim 1 further comprising at least one anti-inflammatory agent.

13. A method for enhancing muscle mass development in animals, comprising:
providing a composition containing a therapeutically effective amount of nicotine and/or nicotinic acetylcholine receptor agonist derived from a natural plant source that provides well-tolerated and ingestible nicotine and/or nicotinic acetylcholine receptor agonist;
administering said composition to an animal; and
subjecting said animal to an exercise regimen following administration of said composition.

14. The method according to claim 13 wherein said animal is a human and said therapeutically effective amount of nicotine and/or nicotinic acetylcholine receptor agonist is from between about 3 μg and about 7 μg.

15. The method according to claim 13 wherein said animal is a small animal weighing between about 30 pounds and 150 pounds, and said therapeutically effective amount of nicotine and/or nicotinic acetylcholine receptor agonist is from between about 10 μg and about 35 μg.

16. The method according to claim 13 wherein said animal is a large animal weighing greater than about 800 pounds, and said therapeutically effective amount of nicotine and/or nicotinic acetylcholine receptor agonist is from between about 22 μg and about 112 μg.

17. The method according to claim 13 wherein said composition is administered from thirty to sixty minutes prior to subjecting said animal to said exercise regimen.

18. The method according to claim 13 wherein said exercise regimen is sufficiently rigorous render a muscle mass of the animal at or near the point of exhaustion.

19. The method according to claim 13 wherein said composition is administered sublingually.

20. A composition for enhancing muscle mass development in a human, comprising:
from about 3 mg to about 9 mg of nicotine and/or nicotinic acetylcholine receptor agonist extract derived from a plant of the Solanaceae family, the extract containing from about 3 μg to about 7 μg of nicotine and/or nicotinic acetylcholine receptor agonist.

21. The composition of claim 20, wherein said plant of the Solanaceae family is green tomato.

22. The composition of claim 20, wherein the composition comprises a dosage form selected from the group consisting of tablet, capsule, and powder.
